(19) United States
(12) Patent Application Publication
(10) Pub. No.: US 2026/0363984 A1
(43) Pub. Date: Nov. 19, 2026

(54) KIT AND METHOD FOR TREATING ALLERGIC CONTACT DERMATITIS AND SEBUM RELATED CONDITIONS

(71) Applicants: David G. Pope, West Newbury, MA (US); James M. Pidhurney, Auburn, NH (US)

(72) Inventors: David G. Pope, West Newbury, MA (US); James M. Pidhurney, Auburn, NH (US)

(21) Appl. No.: 19/746,994

(22) Filed: Jul. 20, 2026

Related U.S. Application Data
(63) Continuation-in-part of application No. 18/464,227, filed on Sep. 10, 2023.
Application No. 18/464,227 is a continuation of application No. 18/149,198, filed on Jan. 3, 2023, now Pat. No. 11,786,449.
Application No. 18/149,198 is a continuation of application No. 18/048,417, filed on Oct. 20, 2022, now Pat. No. 12,521,334.
Application No. 18/048,417 is a continuation-in-part of application No. 17/932,710, filed on Sep. 16, 2022, now Pat. No. 12,383,927.
Application No. 17/932,710 is a continuation-in-part of application No. 17/810,356, filed on Jul. 1, 2022, now Pat. No. 11,660,257.
Application No. 17/932,710 is a continuation-in-part of application No. PCT/US22/73334, filed on Jul. 1, 2022.
(60) Provisional application No. 63/217,686, filed on Jul. 1, 2021.
(60) Provisional application No. 63/217,686, filed on Jul. 1, 2021.
(60) Provisional application No. 63/245,742, filed on Sep. 17, 2021.
(60) Provisional application No. 63/262,117, filed on Oct. 5, 2021.
(60) Provisional application No. 63/262,654, filed on Oct. 18, 2021.
(60) Provisional application No. 63/263,484, filed on Nov. 3, 2021.
(60) Provisional application No. 63/264,674, filed on Nov. 30, 2021.
(60) Provisional application No. 63/854,121, filed on Jul. 30, 2025.

(51) Int. Cl. A61K 8/58; A61J 1/14; A61K 8/04; A61Q 15/00; A61Q 17/00; A63B 23/16; B65D 47/04; B65D 83/06; C01B 33/158; C01B 33/159; C01B 33/18; C09K 3/14

(52) U.S. Cl. CPC A61K 8/585; A61K 8/042; A61Q 15/00; A61Q 17/00; A63B 23/16; B65D 47/04; B65D 83/06; C01B 33/1585; C01B 33/159; C01B 33/18; C09K 3/149; A61J 1/1412; A61K 2800/412; A61K 2800/652; A61K 2800/87; C01P 2004/51; C01P 2004/61

(57) ABSTRACT

A method of skin treatment is provided, in particular a method of treating a skin irritation area by application of a particulate aerogel material. The particulate aerogel material interacts with the skin irritant to create a treatment mixture that, when removed, reduces the volume of skin irritant engaged with skin oil, thereby alleviating symptoms of the skin irritation. In one aspect, the method is used to treat allergic contact dermatitis and sebum-related conditions.

### CROSS-REFERENCE TO RELATED APPLICATIONS

This application claims the benefit of U.S. Provisional Patent Application No. 63/854,121 entitled “Kit and Method for Treating Allergic Contact Dermatitis and Sebum Related Conditions” filed Jul. 30, 2025. This application is also a continuation in part of U.S. patent application Ser. No. 18/464,227 entitled “Particulate Silica Silylate Kit for Treatment of Surfaces for Hydrophobicity Characteristics” filed on Sep. 10, 2023, which is a continuation of U.S. patent application Ser. No. 18/149,198 entitled “Particulate Silica Silylate Kit for Treatment of Surfaces for Hydrophobicity Characteristics” filed on Jan. 3, 2023, which is a continuation of U.S. patent application Ser. No. 18/048,417 entitled “Particulate Silica Silylate Kit for Treatment of Surfaces for Hydrophobicity Characteristics” filed on Oct. 20, 2022, which is a continuation in part of U.S. patent application Ser. No. 17/932,710 entitled “Devices And Methods For Applying A Substance To A Sports Ball” filed on Sep. 16, 2022, which claims the benefit of: U.S. Provisional Patent Application 63/245,742 entitled “Systems And Methods For Applying Particulate Material To Solid Surfaces, Such As Surfaces Of Balls, And Related Articles” filed on Sep. 17, 2021; U.S. Provisional Patent Application No. 63/262,117 entitled “Devices And Methods For Uniform Application Of Aerogel To Sports Balls” Filed on Oct. 5, 2021; U.S. Provisional Patent Application No. 63/262,654 entitled “Devices And Methods For Uniform Application Of Aerogel To Sports Balls” filed Oct. 18, 2021; U.S. Provisional Patent Application No. 63/263,484 entitled “Devices And Methods For Uniform Application Of A Substance To A Sports Ball” filed Nov. 3, 2021; and U.S. Provisional Patent Application No. 63/264,674 entitled “Devices And Methods For Uniform Application Of Aerogel To A Sports Ball” filed Nov. 30, 2021. Furthermore, U.S. patent application Ser. No. 17/932,710 is a continuation-in-part of U.S. patent application Ser. No. 17/810,356 entitled “Particulate Aerogel Material For Grip Enhancement” filed on Jul. 1, 2022, which claims the benefit of U.S. Provisional Application No. 63/217,686 entitled “Particulate Aerogel Material For Grip Enhancement” filed Jul. 1, 2021. Further still, U.S. patent application Ser. No. 17/932,710 is a continuation-in-part of PCT Application No. PCT/US22/73334 filed Jul. 1, 2022, which claims the benefit of U.S. Provisional Application No. 63/217,686. The entire contents of all of the foregoing applications are incorporated herein by reference in their entirety for all purposes.

### FIELD

The disclosure relates generally to methods of skin treatment, and specifically to a method of treating a skin irritation by application of a particulate aerogel material.

### BACKGROUND AND SUMMARY

Conventional methods of treating skin irritations such as allergic contact dermatitis and sebum related conditions are of limited effectiveness and/or of complicated application. The disclosure solves the need to provide an effective and easily applied method to treat skin irritations.

In one embodiment, a method of treating a skin irritation is disclosed, the method comprising: providing a particulate aerogel material; and applying the particulate aerogel material to a skin irritation area, the skin irritation area having a baseline skin irritation value and comprising a skin oil and a skin irritant; wherein: the particulate aerogel material engages at least the skin irritant of the skin irritation area to form a treatment mixture; at least some of the skin irritant is removed from the skin irritation area when at least some of the treatment mixture is removed from the skin irritation area; and the baseline skin irritation value is reduced.

In one aspect, the skin irritant is a dermatitis. The term “dermatitis” means any condition that causes inflammation of the skin, e.g., atopic dermatitis (eczema), contact dermatitis and seborrheic dermatitis (dandruff), commonly causing red rashes, dry skin and itchiness among other symptoms.

In another aspect, the skin irritant is associated with a urushiol exposure. The term “urushiol” means an oily mixture or organic compounds with allergic and sensitizing properties found in particular plants, such as plants in the family anacardiaceae, such as toxicodendron (e.g., poison oak, Chinese lacquer tree, poison ivy, poison sumac), comocladia (e.g., maidenplums), and metopium (poisonwood).

In yet another aspect, the baseline skin irritation value is a measure of the proportion of skin irritant to skin oil.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The terms “determine”, “calculate” and “compute,” and variations thereof, as used herein, are used interchangeably and include any type of methodology, process, mathematical operation or technique.

The term “means” as used herein shall be given its broadest possible interpretation in accordance with 35 U.S.C., Section 112, Paragraph 6. Accordingly, a claim incorporating the term “means” shall cover all structures, materials, or acts set forth herein, and all of the equivalents thereof. Further, the structures, materials or acts and the equivalents thereof shall include all those described in the summary, brief description of the drawings, detailed description, abstract, and claims themselves.

The preceding is a simplified summary of the disclosure to provide an understanding of some aspects of the disclosure. This summary is neither an extensive nor exhaustive overview of the disclosure and its various aspects, embodiments, and/or configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and/or configurations of the disclosure are possible utilizing, alone or in combination, one or more of the features set forth above or described in detail below. Also, while the disclosure is presented in terms of exemplary embodiments, it should be appreciated that individual aspects of the disclosure can be separately claimed.

### BRIEF DESCRIPTION OF THE DRAWINGS

The disclosure will be readily understood by the following detailed description in conjunction with the accompanying drawings, wherein like reference numerals designate like elements. All drawings are scaled drawings. Identical reference numerals have been used, where possible, to designate identical features that are common to the figures.

FIG. 1 depicts a human hand with a skin irritation area;

FIG. 2 is a flow diagram of one method of treating a skin irritation of the disclosure;

FIG. 3 depicts a layer of particulate aerogel material as first applied to a skin irritation area;

FIG. 4 depicts a magnified view of an aerogel particle, according to certain embodiments; and

FIG. 5 depicts a particulate aerogel material of varying size and shape, according to certain embodiments.

It should be understood that the proportions and dimensions (either relative or absolute) of the various features and elements (and collections and groupings thereof) and the boundaries, separations, and positional relationships presented there between, are provided in the accompanying figures merely to facilitate an understanding of the various embodiments described herein and, accordingly, may not necessarily be presented or illustrated to scale, and are not intended to indicate any preference or requirement for an illustrated embodiment to the exclusion of embodiments described with reference thereto.

### DETAILED DESCRIPTION

Reference will now be made in detail to representative embodiments. The following descriptions are not intended to limit the embodiments to one preferred embodiment. To the contrary, it is intended to cover alternatives, modifications, and equivalents as can be included within the spirit and scope of the described embodiments as defined, for example, by the appended claims.

Generally, a method of skin treatment is provided, in particular an unexpected method of treating a skin irritation area by application of a particulate aerogel material. The particulate aerogel material interacts with skin irritant of the skin irritation area to reduce the volume of skin irritant engaged with naturally present skin oil, thereby alleviating symptoms of the skin irritation. In one embodiment, the method is used to treat allergic contact dermatitis and sebum related conditions.

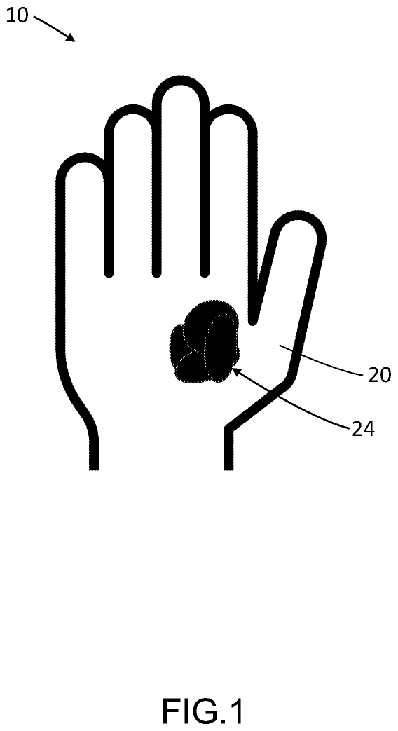

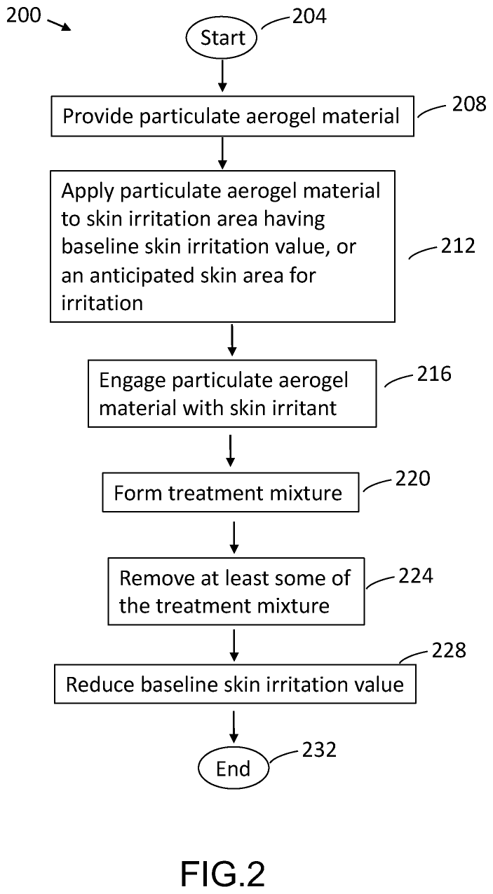

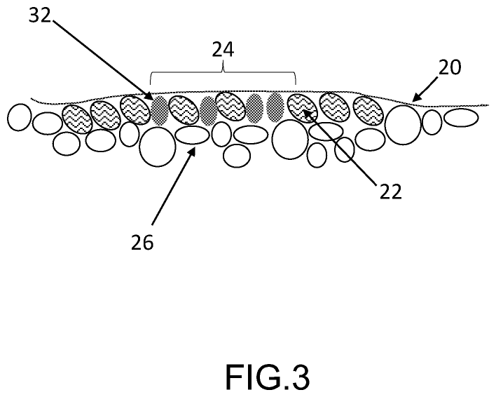

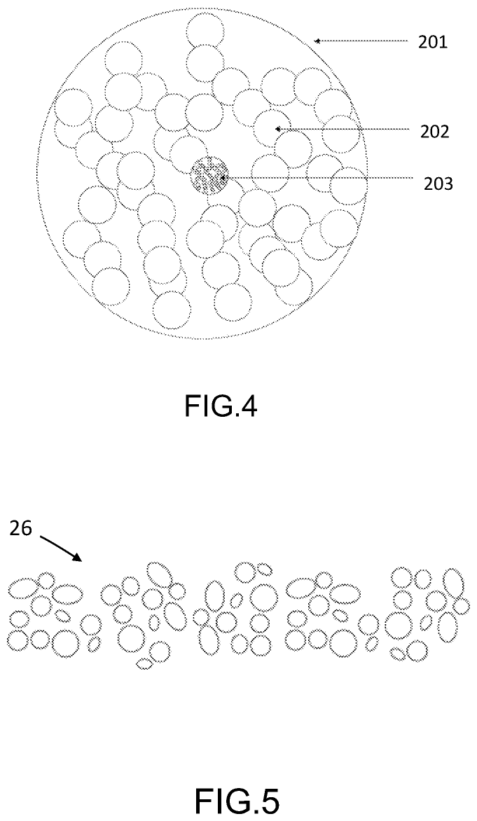

The disclosed methods of use will be described with reference to FIGS. 1-5. FIG. 1 depicts a human hand with a skin irritation area. FIG. 2 is a flow diagram of one method 200 of treating a skin irritation area. FIG. 3 depicts features or aspects of the method 200. FIGS. 4 and 5 depict some features of the aerogel material used in the method 200.

With attention to FIG. 1, a human hand 10 is depicted with a skin irritation area 24 engaged with skin 20 of the human hand 10.

FIG. 2 is a flow diagram of a method 200 of using a treating a skin irritation of the disclosure, such as the skin irritation area 24 of the skin 20 of the human hand 10 described in FIG. 1. In other embodiments, the methods to include method 200 are applied to other areas of a human, such as, without limitation, the arm, shoulder, torso, foot, face, neck, and the like. In some embodiments, the methods to include method 200 are applied to other than a human, such as animals to include, without limitation, canines.

Generally, the method 200 of FIG. 2 starts at step 204 and ends at step 232. The steps are notionally followed in increasing numerical sequence, although, in some embodiments, some steps may be omitted, some steps added, and the steps may follow other than increasing numerical order. The method 200 will be described with reference to FIG. 1 and FIGS. 3-5.

The method 200 starts at step 204 and then proceeds to step 208. At step 208, a particulate aerogel material is provided. The particulate aerogel may be contained in a container adapted to dispense a portion of particulate aerogel. The particulate aerogel material is described in more detail below, to include with respect to FIGS. 3-5. After completing step 208, the method 200 proceeds to step 212.

At step 212, the particulate aerogel material 26 is applied to the skin irritation area 24 of human skin 20, such as the hand 10 depicted in FIG. 1. The particulate aerogel 26 contacts or engages with or interacts with the skin irritation area 24. In certain embodiments, the particulate aerogel material 26 is applied to a skin area that is anticipated to be exposed to a skin irritant but before there is any skin irritation.

When the skin 20 is exposed to a skin irritant 32, the skin irritant 32 interacts with or engages with or intermingles with the skin oil 22 to form the skin irritation area 24, as depicted in FIGS. 1 and 3. The human skin 20 comprises skin oil 22, as depicted in FIG. 3, which generally is disposed or positioned on the exterior surface of the skin 20.

The skin irritant 32 may be from any of a number of sources to include those associated with a urushiol exposure. In most individuals, urushiol causes an allergic skin rash (i.e., a skin irritation area) on contact, known as urushiol-induced contact dermatitis. As depicted in FIG. 3, the skin irritation area 24 comprises comingled skin oil 22 and skin irritant 32. Healthy skin 20 comprises skin oil 22 but does not comprise skin irritant 32.

Note that the skin irritant may be any of a number of foreign or unwelcomed substances that cause skin irritation, to include those associated with a urushiol exposure and urushiol-induced contact dermatitis, and further to include skin related health issues associated with sebum (i.e., yeast thriving in skin oil, e.g., seborrheic dermatitis, tinea versicolor) and afflictions caused by transient plant based oils being deposited on the body (e.g., urushiol exposure such as poison ivy, poison oak, poison sumac, etc.), synthetic oil exposure (e.g., industrial oils, lubricants, fragrances, etc.), or acne.

The skin irritation area 24 has a skin irritation value. When the skin 20 is uncompromised or an otherwise natural and healthy state, the skin irritation value is at its lowest value, e.g., zero. This value is deemed the normal skin irritation value.

However, when the skin 20 is in an atypical or unhealthy state when suffering from a skin irritation and having at least one skin irritation area 24, the skin irritation value is greater than the normal skin irritation value and is deemed a baseline skin irritation value. Upon application or execution of the method 200, the skin irritation value is reduced from the baseline skin irritation value.

The skin irritation value (the “value”) may be determined or established in any number of ways. As one non-limiting example, the value may be calculated based on a percentage of a volume of the skin irritant 32 to a volume of the skin oil 22 in the skin irritation area 24. Or, as another non-limiting example, the value may be calculated as the percentage of surface area of the skin irritation area 24 relative to the unaffected/healthy area of the skin (e.g., in the FIG. 1 example, a percentage of the irritation area 24 relative to the remaining healthy area of the hand's palm). After completing step 212, the method 200 proceeds to step 216.

At step 216, the particulate aerogel 26 contacts or engages with or interacts with the skin irritant 32 of the skin irritation area 24 and/or the skin oil 22. Various interfaces or interactions or engagements of the particulate aerogel 26 with the skin irritant 32 and the skin oil 22 are depicted in FIG. 3. Specifically, the particulate aerogel 26 is shown engaging the skin irritation area 24 comprising skin irritant 32 and skin oil 22, and also engaging healthy areas of the skin 20 that comprise skin oil 22 without skin irritant 32 (as shown to the right of the skin irritation area 24 in FIG. 3). After completing step 216, the method 200 proceeds to step 220.

At step 220, the particulate aerogel 26 forms a treatment mixture with at least some of the skin irritant 32 of the skin irritation area 24 to form an aerogel-irritant mixture. In some embodiments, the treatment mixture comprises particulate aerogel 26 and skin irritant 32. In some embodiments, the treatment mixture comprises particulate aerogel 26, skin irritant 32, and skin oil 22. (The term “mixture” means when two or more substances mix with each other without participating in a chemical change). After completing step 220, the method 200 proceeds to step 224.

At step 224, at least some of the at least some of the treatment mixture is removed from the skin irritation area 24, thereby removing at least some of the skin irritant 32 from the skin irritation area 24. Stated another way, the particulate aerogel 26 thus operates to strip away the skin irritant 32 (e.g., harmful oils) immediately after exposure and also once symptoms have presented themselves, thus containing the spread and alleviating the symptoms associated with the contaminant or infection. After completing step 224, the method 200 proceeds to step 228. In certain embodiments, step 224 is an optional step.

At step 228, as a result of the removal of at least some of the skin irritant 32 by way of removal of the treatment mixture results in a lowering or a reduction on the baseline skin irritation value. After completing step 228, the method 200 proceeds to step 232 and the method 200 ends.

FIG. 4 depicts a magnified view of an aerogel particle, according to certain embodiments. The schematic shows an aerogel particle 201 showing the primary porosity 202 and secondary porosity 203. In some embodiments, the aerogel particle comprises an inorganic oxide aerogel. In certain embodiments, the inorganic oxide aerogel comprises an oxide of silicon, aluminum, titanium, hafnium, zirconium, chromium, niobium, tantalum, iron, vanadium, neodymium, samarium, holmium, zinc, magnesium, calcium, and/or erbium. In some preferred embodiments the inorganic oxide aerogel comprises silica. In further preferred embodiments, the silica comprises a hydrophobic silica.

FIG. 5 depicts a particulate aerogel material 26 of varying size and shape, according to certain embodiments. In one embodiment, the particulate aerogel material is applied to a skin irritation area such that the average layer thickness is greater than or equal to 50 nanometers, and less than or equal to 1 millimeter. In some embodiments when applied to a human hand, the particulate aerogel material can cover at least 25%, at least 40%, at least 50%, at least 75%, at least 90%, at least 95%, at least 99% or all of the interior surface area of a human hand. In some embodiments, the particulate aerogel material can form a conformal coating over at least 25%, at least 40%, at least 50%, at least 75%, at least 90%, at least 95%, at least 99% or all of the interior surface area of a human hand. Further characteristics and embodiments of the particulate aerogel will now be described.

In some embodiments the particulate aerogel material exhibits a high surface area. In some embodiments, the particulate aerogel material exhibits a surface area of at least 50 m2/g, at least 100 m2/g, at least 200 m2/g, at least 300 m2/g, at least 400 m2/g, at least 500m2/g, at least 600 m2/g, at least 700 m2/g, at least 800 m2/g, at least 900 m2/g, at least 1000 m2/g, at least 1100 m2/g, or at least 1200 m2/g.

In some embodiments, the particulate aerogel material exhibits a highly porous structure. In some embodiments, the particulate aerogel material exhibits a BJH pore volume of greater than or equal to 0.05 g/cm3 , greater than or equal to 1 g/cm3, greater than or equal to 2 g/cm3 , greater than or equal to 3 g/cm3 , greater than or equal to 4 g/cm3, or less than or equal to 5 g/cm3 . In some embodiments, the particulate aerogel material exhibits a small pore size. In some embodiments, the particulate aerogel material exhibits a BJH average pore width of less than 10 nm, less than 20 nm, less than 30 nm, less than 40 nm, less than 50 nm, less than 60 nm, less than 70 nm, less than 80 nm, less than 90 nm, less than 100 nm, less than 500 nm, less than 1 μm, less than 10 μm, less than 100 μm, or less than 1 mm. In some embodiments, the high pore volume of the particulate aerogel material results in good sorption. In some embodiments, the small pore size of the particulate aerogel material results in efficient capillarity driven wetting of natural oils found on human skin, and/or perspiration.

In some embodiments, the particulate aerogel material has an average maximum cross-sectional dimension. In some embodiments, the average maximum cross-sectional dimension of the particulate aerogel material is less than or equal to 1 centimeter. In some embodiments, the average maximum cross-sectional dimension of the particulate aerogel material is greater than or equal to 50 nanometers and less than or equal to 1 centimeter. In some embodiments, the average maximum cross-sectional dimension of the particulate aerogel is greater than or equal to 50 nm, greater than or equal to 100 nm, greater than or equal to 250 nm, greater than or equal to 500 nm, greater than or equal to 1 micrometer, greater than or equal to 10 micrometers, greater than or equal to 25 micrometers, greater than or equal to 50 micrometers, greater than or equal to 100 micrometers, greater than or equal to 250 micrometers, greater than or equal to 500 micrometers, greater than or equal to 1 millimeter, or greater than or equal to 1 centimeter. The average maximum cross-sectional dimension is taken as a number average and can be measured using microscopy. In some embodiments, the average maximum cross-sectional dimension of the particulate aerogel material can be determined by placing a representative sample of the particulate aerogel material on a slide or other suitable analysis substrate, imaging the particles (e.g., using image capture hardware and software to capture an image of the particulate aerogel material sample under proper magnification), and then determining the largest cross-sectional dimension of each particle (e.g., using an image processing software to find the maximum cross-sectional dimensions of each discrete particle present in the sample). Suitable magnification devices include an optical microscope or a scanning electron microscope (SEM). The maximum cross-sectional dimensions of all discrete particles are then averaged to determine the average maximum cross-sectional dimension of the sample.

In some embodiments, at least 50 vol % of the particulate aerogel material is made up of particles having a maximum cross-sectional dimension of less than or equal to 1 centimeter. In some embodiments, at least 50 vol % (or at least 60 vol %, at least 70 vol %, at least 80 vol %, at least 90 vol %, or at least 95 vol %) of the particulate aerogel material is made up of particles having a maximum cross-sectional dimension of greater than or equal to 50 nanometers, greater than or equal to 0.1 mm, greater than or equal to 0.7 mm, greater than or equal to 1 mm, and/or less than or equal to 1 centimeter, less than or equal to 3 mm, or less than or equal to 1.2 mm. In some embodiments, at least 50 vol %, at least 60 vol %, at least 70 vol %, at least 80 vol %, at least 90 vol %, or at least 95 vol % of the particulate aerogel material is made up of particles having a maximum cross-sectional dimension of less than or equal to 1 centimeter. In some embodiments, at least 50 vol % of the particulate aerogel material is made up of particles having a maximum cross-sectional dimension of greater than or equal to 50 nanometers and less than or equal to 1 centimeter. In some embodiments, at least 50 vol %, at least 60 vol %, at least 70 vol %, at least 80 vol %, at least 90 vol %, or at least 95 vol % of the particulate aerogel material is made up of particles having a maximum cross-sectional dimension of greater than or equal to 50 nanometers and less than or equal to 1 centimeter. In some embodiments, at least 50 vol %, at least 60 vol %, at least 70 vol %, at least 80 vol %, at least 90 vol %, or at least 95 vol % of the particulate aerogel material is made up of particles having a maximum cross-sectional dimension of greater than or equal to 0.1 mm and less than or equal to 1.2 mm. In certain preferred embodiments, at least 50 vol %, at least 60 vol %, at least 70 vol %, at least 80 vol %, at least 90 vol %, or at least 95 vol % of the particulate aerogel material is made up of particles having a maximum cross-sectional dimension of greater than or equal to 1 mm and less than or equal to 3 mm. In certain preferred embodiments at least 50% of the particulate aerogel material is made up of particles having a maximum cross-sectional dimension of greater than or equal to 0.7 mm and less than or equal to 1.2 mm. In certain preferred embodiments, at least 50 vol %, at least 60 vol %, at least 70 vol %, at least 80 vol %, at least 90 vol %, or at least 95 vol % of the particulate aerogel material is made up of particles having a maximum cross-sectional dimension of greater than or equal to 0.7 mm and less than or equal to 1.2 mm. In certain preferred embodiments, at least 50 vol %, at least 60 vol %, at least 70 vol %, at least 80 vol %, at least 90 vol %, or at least 95 vol % of the particulate aerogel material is made up of particles having a maximum cross-sectional dimension of greater than or equal to 0.1mm and less than or equal to 4 mm.

In some embodiments, at least 50 vol % (or at least 60 vol %, at least 70 vol %, at least 80 vol %, at least 90 vol %, or at least 95 vol %) of the particulate aerogel material is made up of particles having an ISO 13320-1 cross-sectional dimension of greater than or equal to 50 nanometers, greater than or equal to 0.1 mm, greater than or equal to 0.7 mm, greater than or equal to 1 mm, and/or less than or equal to 1 centimeter, less than or equal to 3 mm, or less than or equal to 1.2 mm. The “ISO 13320-1 cross-sectional dimension,” as used herein, refers to the largest cross-sectional dimension of the particulate aerogel when measured by laser diffraction according to the standard ISO 13320-1. In certain preferred embodiments, at least 50 vol %, at least 60 vol %, at least 70 vol %, at least 80 vol %, at least 90 vol %, or at least 95 vol % of the particulate aerogel material is made up of particles having an ISO 13320-1 cross-sectional dimension of greater than or equal to 0.7 mm and less than or equal to 1.2 mm.

In some embodiments, the particulate aerogel material comprises an inorganic oxide aerogel. In some embodiments, at least 50 wt %, at least 60 wt %, at least 70 wt %, at least 80 wt %, at least 90wt %, at least 95 wt %, at least 99 wt %, at least 99.9 wt %, or at least 99.99 wt % of the particulate aerogel material is made of inorganic oxide aerogel. In some embodiments the inorganic oxide aerogel comprises an oxide of silicon, aluminum, titanium, hafnium, zirconium, chromium, niobium, tantalum, iron, vanadium, neodymium, samarium, holmium, zinc, magnesium, calcium, and/or erbium. In some preferred embodiments, the inorganic oxide aerogel comprises silica aerogel (e.g., in an amount of at least 50 wt %, at least 60 wt %, at least 70 wt %, at least 80 wt %, at least 90wt %, at least 95 wt %, at least 99 wt %, at least 99.9 wt %, or at least 99.99 wt %). In further preferred embodiments, the inorganic oxide aerogel comprises trimethylsilylated silica aerogel (e.g., in an amount of at least 50 wt %, at least 60 wt %, at least 70 wt %, at least 80 wt %, at least 90wt %, at least 95 wt %, at least 99 wt %, at least 99.9 wt %, or at least 99.99 wt %).

In some embodiments, the particulate aerogel material may exhibit an internal specific surface area. As used herein, the internal surface area and specific surface area have the same meaning and describe the same phenomenon. As described herein, these values may also be referred to as the BET surface area. The internal specific surface area of a particulate aerogel material may be determined using nitrogen adsorption porosimetry and deriving the surface area value using the Brunauer-Emmett-Teller (BET) model. For example, nitrogen sorption porosimetry may be performed using a Micromeritics Tristar II 3020 surface area and porosity analyzer. Before porosimetry analysis, specimens may be subjected to vacuum of ~100 torr for 24 hours to remove adsorbed water or other solvents from the pores of the specimens. The porosimeter may provide an adsorption isotherm and desorption isotherm, which show the amount of analyte gas adsorbed or desorbed as a function of partial pressure. Specific surface area may be calculated from the adsorption isotherm using the BET method over ranges typically employed in measuring surface area. In some embodiments, the BET surface area of the particulate aerogel material is greater than or equal to 5 m2/g, greater than or equal to 50 m2/g, greater than or equal to 100 m2/g, greater than or equal to 200 m2/g, greater than or equal to 300 m2/g, greater than or equal to 400 m2/g, greater than or equal to 500 m2/g, greater than or equal to 600 m2/g, greater than or equal to 700 m2/g, greater than or equal to 800 m2/g, greater than or equal to 1000 m2/g, greater than or equal to 2000 m2/g, greater than or equal to 3000 m2/g, and/or less than or equal to 1500 m2/g, or less than or equal to 4000 m2/g. In some embodiments, the BET surface area of the particulate aerogel material is greater than or equal to 5 m2/g and less than or equal to 4000 m2/g. In certain preferred embodiments, the BET surface area of the particulate aerogel material is greater than or equal to 100 m2/g and less than or equal to 1500 m2/g. Values of the BET surface area of the aerogel outside of these ranges may be possible. In some preferred embodiments, the particulate aerogel material exhibits a BET surface area greater than 200 m2/g. In further preferred embodiments, the particulate aerogel material exhibits a BET surface area greater than 500 m2/g.

The particulate aerogel material may have any of a variety of suitable pore structures. Pore width distribution, pore area distribution, and mean pore size may be calculated from the nitrogen desorption isotherm using the Barrett-Joyner-Halenda (BJH) method over ranges typically reemployed in measuring pore width and pore area distribution. In some embodiments, the particulate aerogel material comprises pores of less than or equal to 100 microns, less than or equal to 10 microns, less than or equal to 1 micron, less than or equal to 500 nm, less than or equal to 250 nm, less than or equal to 100 nm, less than or equal to 50 nm, less than or equal to 25 nm, less than or equal to 20 nm, less than or equal to 15 nm, and/or less than or equal to 10 nm. In some embodiments the aerogel comprises pores of greater than or equal to 10 nm, greater than or equal to 15 nm, greater than or equal to 20 nm, greater than or equal to 25 nm, greater than or equal to 50 nm, greater than or equal to 100 nm, greater than or equal to 250 nm, greater than or equal to 500 nm, greater than or equal to 1 micron, greater than or equal to 10 microns, and or greater than or equal to 100 microns. Average pore width, e.g., mean pore size, (assuming cylindrical pores) may be calculated using pore width=4*(total specific volume)/(specific surface area) where total specific volume and specific surface area may also be calculated using BJH analysis of the desorption isotherm. In some embodiments, the average pore width is less than or equal to 10 nm, less than or equal to 20 nm, less than or equal to 30 nm, less than or equal to 40 nm, less than or equal to 50 nm, less than or equal to 60 nm, less than or equal to 70 nm, less than or equal to 80 nm, less than or equal to 90 nm, less than or equal to 100 nm, less than or equal to 500 nm, less than or equal to 1 μm, less than or equal to 10 μm, less than or equal to 100 μm, or less than or equal to 1 mm. In certain preferred embodiments, the average pore width of the particulate aerogel material is less than or equal to 50 nm. In some preferred embodiments, the average pore width of the particulate aerogel material is less than or equal to 20 nm.

In some embodiments, the pore width distribution of the aerogel may be unimodal (i.e., exhibiting a single maximum). In some embodiments, the pore width distribution maximum is found at less than or equal to 10 nm, less than or equal to 20 nm, less than or equal to 30 nm, less than or equal to 40 nm, less than or equal to 50 nm, less than or equal to 60 nm, less than or equal to 70 nm, less than or equal to 80 nm, less than or equal to 90 nm, less than or equal to 100 nm, less than or equal to 500 nm, less than or equal to 1 μm, less than or equal to 10 μm, less than or equal to 100 μm, or less than or equal to 1 mm. In some embodiments, the aerogel comprises a unimodal pore size distribution.

In some embodiments, the pore width distribution of the aerogel may be bimodal, or at least bimodal. In some embodiments, the aerogel material can have two distinct populations of pores, one with an average pore size less than a certain critical pore width, and one with an average pore size greater than some critical pore width. In some embodiments, the critical pore width is less than or equal to 10 nm, less than or equal to 20 nm, less than or equal to 30 nm, less than or equal to 40 nm, less than or equal to 50 nm, less than or equal to 60 nm, less than or equal to 70 nm, less than or equal to 80 nm, less than or equal to 90 nm, less than or equal to 100 nm, less than or equal to 500 nm, less than or equal to 1 μm, less than or equal to 10 μm, less than or equal to 100 μm, or less than or equal to 1 mm. In some embodiments, the aerogel comprises a bimodal pore size distribution.

In some embodiments, the particulate aerogel material exhibits a BJH pore volume of greater than or equal to 0.05 cm3/g and less than or equal to 5 cm3/g. In some embodiments, the particulate aerogel material exhibits a BJH pore volume of greater than or equal to 0.05 g/cm3 , greater than or equal to 1 g/cm3, greater than or equal to 2 g/cm3, greater than or equal to 3 g/cm3 , greater than or equal to 4 g/cm3 , and/or less than or equal to 5 g/cm3 .

In some embodiments, the particulate aerogel material is mixed with a water-based or solvent-based carrier to create a cream or paste. This liquid-dispersed aerogel is applied and worked into the skin to remove the oil—natural or transient or both. The liquid carrier may include additives that help to sooth the affected skin.

In some embodiments, the particulate aerogel material may be present on the skin for a period of time greater than or equal to 1 second, greater than or equal to 1 minute, greater than or equal to 15 minutes, greater than or equal to 30 minutes, greater than or equal to 1 hour, greater than or equal to 4 hours, or greater than or equal to 8 hours. In some embodiments, greater than or equal to 1%, greater than or equal to 5%, greater than or equal to 10%, greater than or equal to 20%, greater than or equal to 30%, greater than or equal to 40%, greater than or equal to 50%, greater than or equal to 60%, greater than or equal to 70%, greater than or equal to 80%, greater than or equal to 90%, or greater than or equal to 95% of the adhered particulate aerogel material may remain on human skin after vigorous exercise. In some embodiments, vigorous exercise may be described as a physical activity lasting more than 15 minutes during which the heart rate increases to at least 120 beats per minute for that period of time during said physical activity. In some embodiments, greater than or equal to 1%, greater than or equal to 5%, greater than or equal to 10%, greater than or equal to 20%, greater than or equal to 30%, greater than or equal to 40%, greater than or equal to 50%, greater than or equal to 60%, greater than or equal to 70%, greater than or equal to 80%, greater than or equal to 90%, or greater than or equal to 95% of the adhered particulate aerogel material may remain on human skin after the particulate aerogel material coated human skin is submerged in water for a period of 5 minutes. In some embodiments, greater than or equal to 1%, greater than or equal to 5%, greater than or equal to 10%, greater than or equal to 20%, greater than or equal to 30%, greater than or equal to 40%, greater than or equal to 50%, greater than or equal to 60%, greater than or equal to 70%, greater than or equal to 80%, greater than or equal to 90%, or greater than or equal to 95% of the adhered particulate aerogel material may remain on human skin after the particulate aerogel material coated human skin contacts a solid surface. Percent of adhered particulate aerogel material remaining on human skin may be determined by visual inspection, optical microscopy, or qualitative touch analysis of the coated human skin.

In some embodiments, the particulate aerogel material is non-toxic to humans. In some embodiments, the particulate aerogel material is non-carcinogenic to humans.

In some embodiments, the particulate aerogel material is non-marking. The particulate aerogel material is considered to be non-marking if the particulate aerogel material does not leave behind a visibly observable permanent deposit of material after contacting a solid surface.

Aerogels are a class of low-density solid materials comprising a porous three-dimensional network of interconnected nanostructures. Aerogels typically exhibit a wide array of desirable materials properties including high specific surface area, low bulk density, low thermal conductivity, and/or low dielectric constant, among others. Aerogels are typically produced through the sol-gel process. Briefly, in such processes, monomers are reacted in a solution to form a dispersion of nanoparticles in liquid called a sol. Alternatively, nanoparticles can be dispersed in a solution to make a sol and in a second step or concurrently with the first step, the particles form a gel. The pore fluid of that gel is then extracted in such a way as to prevent substantial densification leaving behind a highly porous solid (an aerogel). Inorganic oxide aerogels are, generally, natively hydrophilic and express hydroxylated surface functionality that absorbs moisture. Alternatively, inorganic oxide aerogel materials can be made hydrophobic by reacting either the gel in its gel phase, or the dry aerogel, with a reactive hydrophobe. A hydrophobe is generally a reactive chemical agent used to impart hydrophobicity unto a gel or aerogel by changing the composition of surface functional groups that line the backbone of said gel or aerogel. The gel can be reacted with a hydrophobe by soaking in a solution comprising the hydrophobe. The aerogel can be reacted with a hydrophobe by flowing a vapor containing the hydrophobe through the pores of the aerogel. In some embodiments, the hydrophobe may comprise an organosilane. In some preferred embodiments, the hydrophobe comprises hexamethyldisiloxane. In some preferred embodiments, the hydrophobe comprises hexamethyldisilane. In some embodiments, the hydrophobic particulate aerogel material comprises trimethylsilylated silica aerogel.

In some embodiments, particulate aerogel material can be made by first manufacturing a larger aerogel monolith and subsequently breaking the monolith down into particulate matter.

A “partition coefficient” (P) of a compound is the ratio of concentrations of the compound in a mixture of n-octan-1-ol and water at equilibrium. “Log P” of the compound is the logarithm (Log) of the compound's partition coefficient. The compound's Log P is determined according to the equation below:

Log P=Log ((Concentration of the compound in the n-octan-1-ol phase of the mixture)/(Concentration of the compound in the aqueous phase of the mixture)),

e.g., when the compound is not ionized in n-octan-1-ol and water. Log P may be determined at about 25□ C. and about 1 atm. A higher Log P value may suggest a higher hydrophobicity. In some embodiments, a hydrophobe may have a Log P of greater than or equal to 1, greater than or equal to 1.5, greater than or equal to 2, greater than or equal to 2.5, greater than or equal to 3, greater than or equal to 3.5, or greater than or equal to 4.

In some embodiments, after reacting an inorganic oxide aerogel with a suitable hydrophobe, the Log P of the aerogel is increased. In some embodiments, after reacting an inorganic oxide aerogel with a suitable hydrophobe, the Log P of the reacted aerogel is increased by greater than or equal to 0.5 points, greater than or equal to 1 point, greater than or equal to 1.5 points, greater than or equal to 2 points, greater than or equal to 2.5 points, or greater than or equal to 3 points. In some embodiments, the Log P of the reacted inorganic oxide aerogel is greater than or equal to 1, greater than or equal to 2, greater than or equal to 2.5, greater than or equal to 3, greater than or equal to 3.5, or greater than or equal to 4.

In some embodiments, the aerogels described herein may have a Log P of greater than or equal to 1, greater than or equal to 1.5, greater than or equal to 2, greater than or equal to 2.5, greater than or equal to 3, greater than or equal to 3.5, or greater than or equal to 4.

In some embodiments, the particulate aerogel material may exhibit hydrophobicity.

The term hydrophobicity refers to the absence and/or partial absence of attractive force between a material and a mass of water. In some embodiments, the hydrophobicity of a bulk material refers to this behavior as it applies to a surface. In certain embodiments, the apparent hydrophobicity of a textured surface can be higher than the chemical hydrophobicity of the bulk material.

Hydrophobicity of the particulate aerogel material can be expressed in terms of the liquid water uptake. The term liquid water uptake refers to the ability of a material or composition to absorb, adsorb, or otherwise retain water due to contact with water in the liquid state. Liquid water uptake can be expressed one of several ways, for example, as a fraction or percent of the open pore volume or envelope volume of the particulate aerogel material, or as a fraction or percent relative to the mass of the unwetted particulate aerogel material. The liquid water uptake reported is understood to be a measurement undertaken under specific conditions. A particulate aerogel material that has superior or improved liquid water uptake relative to a different particulate aerogel material is understood to have a lower uptake of liquid water.

In some embodiments, the particulate aerogel material has a liquid water uptake of less than 100 wt %, less than 80 wt %, less than 70 wt %, less than 60 wt %, less than 50 wt %, less than 40 wt %, less than 30 wt %, less than 20 wt %, less than 10 wt %, less than 5 wt %, less than 4 wt %, less than 3 wt %, less than 2 wt %, less than 1 wt %, or less than 0.1 wt % relative to the weight of the particulate aerogel material before contact with liquid water when measured according to standard ASTM C1763.

In some embodiments, the particulate aerogel material has a liquid water uptake of less than 100 wt %, less than 80 wt %, less than 70 wt %, less than 60 wt %, less than 50 wt %, less than 40 wt %, less than 30 wt %, less than 20 wt %, less than 10 wt %, less than 5 wt %, less than 4 wt %, less than 3 wt %, less than 2 wt %, less than 1 wt %, or less than 0.1 wt % relative to the weight of the particulate aerogel material before contact with liquid water when measured according to standard EN 1609.

In some embodiments, when the particulate aerogel material is submerged under water at 25° C. for 24 h, the particulate aerogel material uptakes a mass of water within its outer boundaries of less than 30% of the dry mass of the particulate aerogel material prior to submerging in the water.

Hydrophobicity of the particulate aerogel material can be expressed in terms of the water contact angle. The term water contact angle refers to the equilibrium contact angle of a drop of water in contact with a surface made of the particulate aerogel material. The contact angle can be determined as follows: A polycarbonate annulus with a 2 cm outer diameter and a 1 cm inner diameter, that is 2 cm tall and sealed on one end by a flat piece of polycarbonate forming a 2 cm deep well is prepared. Particulate aerogel material is added to the top of the well. The polycarbonate cylinder is then tapped 10 times to allow the particulate aerogel material to settle. More particulate aerogel material is added, and this process is repeated until the level of particulate in the polycarbonate cylinder is level with or above the top of the well. A metal laboratory spatula is then dragged across the top of the well to scrape away excess material and help ensure the flatness of the testing area. A droplet of deionized water is them deposited via Pasteur pipette atop the center of the particulate aerogel material. A photo of the droplet of water is then captured from the side of the droplet. The image of the droplet is then processed using an image processing software to measure the contact angle between the droplet and the top surface of the particulate in the well, where the contact angle is measured through the bulk of the droplet. In some embodiments, the particles are milled or crushed prior to testing such that the average maximum cross-sectional dimension of the aerogel particles is greater than or equal to 2 micrometers and less than or equal to 40 micrometers. Without wishing to be bound by any particular theory, it is believed that, in some embodiments, a smaller particle size allows for tighter packing of the particulate aerogel material to reduce textural surface effects on the contact angle measurement. Without wishing to be bound by any particular theory, it is believed that a particulate aerogel that exhibits a higher water contact angle relative to a second particulate aerogel material may have a superior or improved hydrophobicity relative to that second particulate aerogel material. In some embodiments, when the particulate aerogel material is tested according to this method, the water contact angle may be greater than 90°, greater than 100°, greater than 110°, greater than 120°, greater than 130°, greater than 140°, greater than 150°, greater than 160°, greater than 170°, or between 170° and 180° in ambient air at least one temperature and pressure. In some embodiments, when the particulate aerogel material is tested according to this method, the water contact angle may be greater than 90°, greater than 100°, greater than 110°, greater than 120°, greater than 130°, greater than 140°, greater than 150°, greater than 160°, greater than 170°, or between 170° and 180° in ambient air at 1 atm pressure and 25° C. In some preferred embodiments, the particulate aerogel material exhibits a contact angle with water, in an ambient air environment at 1 atm and 25° C., of greater than 90° when measured according to the test described herein.

Hydrophobicity of the particulate aerogel material can be expressed in terms of the water vapor uptake. The term water vapor uptake refers to the ability for a material or composition to absorb, adsorb, or otherwise retain water due to contact with water in the vapor state. Water vapor uptake can be expressed as a fraction or percent of water retained relative to the mass of the particulate aerogel material before exposure to water vapor. The water vapor uptake reported is understood to be a measurement undertaken under specific conditions. A particulate aerogel material which has superior or improved water vapor uptake relative to a different particulate aerogel material is understood to have a lower sorption or retention of water vapor. In some embodiments, the water uptake may be less than 100 wt %, less than 80 wt %, less than 70 wt %, less than 60 wt %, less than 50 wt %, less than 40 wt %, less than 30 wt %, less than 20 wt %, less than 10 wt %, less than 5 wt %, less than 4 wt %, less than 3 wt %, less than 2 wt %, less than 1 wt %, or less than 0.1 wt % relative to the weight of the particulate aerogel material before exposure to water vapor, when measured according to standard ASTM C1104.

Hydrophobicity of the particulate aerogel material can be expressed in terms of retained internal surface area after submersion in water. After submersion in deionized water, the particulate aerogel material is allowed to dry out completely, and then nitrogen sorption porosimetry is performed as outlined herein to measure the remaining internal surface area of the material. In some embodiments, the particulate aerogel material retains greater than 5%, greater than 10%, greater than 15%, greater than 20%, greater than 25%, greater than 30%, greater than 40%, greater than 50%, greater than 60%, greater than 70%, greater than 80%, greater than 90%, or greater than 95% of the internal surface area of the same particulate aerogel material prior to submersion in water.

In some embodiments the particulate aerogel material exhibits an optical transmission of greater than or equal to 5% at 635 nm. In some embodiments, the particulate aerogel material may exhibit a relatively high light transmission. In some embodiments, the particulate aerogel material exhibits a light transmission of greater than or equal to 5%, greater than or equal to 10%, greater than or equal to 20%, greater than or equal to 30%, greater than or equal to 40%, greater than or equal to 50%, greater than or equal to 60%, greater than or equal to 70%, greater than or equal to 80%, greater than or equal to 90%, or greater than or equal to 95% when tested according to ASTM E424. In some preferred embodiments, the particulate aerogel material exhibits a light transmission of greater than or equal to 89% per cm when tested according to ASTM E424.

In some embodiments, the particulate aerogel material readily absorbs natural oils from human skin. In some embodiments, the particulate aerogel material exhibits a mineral oil uptake that can serve as a proxy test for gauging the particulate aerogel material's ability to absorb natural oil excreted from human skin. The term mineral oil uptake refers to the ability of a material or composition to absorb, adsorb, or otherwise retain mineral oil due to contact with mineral oil. Mineral oil uptake can be expressed as a fraction or percent relative to the mass of the unwetted particulate aerogel material. The mineral oil uptake described herein is measured using CAS number 8012-95-1 mineral oil from Sigma Aldrich. A particulate aerogel material that has superior or improved mineral oil uptake relative to a different particulate aerogel material is understood to have a higher uptake of mineral oil. In some embodiments, the mineral oil uptake may be greater than or equal to 0.1 wt %, greater than or equal to 1 wt %, greater than or equal to 2 wt %, greater than or equal to 3 wt %, greater than or equal to 4 wt %, greater than or equal to 5 wt %, greater than or equal to 10 wt %, greater than or equal to 20 wt %, greater than or equal to 30 wt %, greater than or equal to 40 wt %, greater than or equal to 50 wt %, greater than or equal to 60 wt %, greater than or equal to 70 wt %, greater than or equal to 80 wt %, greater than or equal to 100 wt %, greater than or equal to 200 wt %, greater than or equal to 300 wt %, greater than or equal to 500 wt %, or greater than or equal to 1000 wt % relative to the weight of the particulate aerogel material before submersion in mineral oil.

In some embodiments, the particulate aerogel material is submerged under mineral oil for 24 hours at 25 C, the particulate aerogel material uptakes a mass of mineral oil within the outer boundaries of the particulate of greater than or equal to 20% of the dry mass of the particulate aerogel material just prior to submerging in mineral oil.

In some embodiments, the particulate aerogel material will fracture when subjected to a pressure of less than 1 N/cm2. In some embodiments, the particulate aerogel material will fracture when subjected to a pressure of less than or equal to 10 kPa, less than or equal to 20 kPa, less than or equal to 50 kPa, less than or equal to 100 kPa, or less than or equal to 200 kPa. The pressure at which the particulate aerogel material will fracture can be measured according to the following. An optically transparent polycarbonate annulus with a 2 cm outer diameter and a 1 cm inner diameter, that is 2 cm tall and sealed on one end by a flat piece of polycarbonate forming a 2 cm deep well, said well having a level marking on the side of its outer surface corresponding to a well depth of 1 cm, is prepared. Particulate aerogel material is added to the level line within the well. The polycarbonate well is then tapped 10 times to allow the particulate aerogel material to settle. More particulate aerogel material is added, and this process is repeated until the level of particulate in the polycarbonate well is exactly at the 1 cm depth level mark. The polycarbonate annulus is then centered between the compressive platens on a universal testing machine (e.g., an Instron 3366). A 0.9 cm diameter polycarbonate rod that is 2 cm tall is gently placed on top of the aerogel in the well of the polycarbonate annulus. The compressive platens are then compressed at a rate of 1 mm/s and the force is recorded as a function of displacement, until a force of 0.64 N is reached, corresponding to a pressure exerted by the cylinder of 200 kPa, or the 2 cm rod can no longer be depressed. The compressive platens are then returned to their starting positions, to remove the external load on the volume of aerogel. The material is stated to be crushable if a stress of less than or equal to 200 kPa results in the breaking of at least one particle in the well into two or more pieces as determined through visual observation before and after the test. In some embodiments, permanent compaction of the volume of the particulate aerogel material in the well results from the test described herein, such that there is a permanent compressive axial deflection of the volume of particles in an amount greater than 5% after a stress of 200 kPa has been applied. In some embodiments, the particulate aerogel material will fracture when subjected to a pressure of less than 1 N/cm2.

In some embodiments, a kit is provided. A “kit,” as used herein, typically defines a package or an assembly including one or more of the components disclosed herein, and/or other components associated with the embodiments disclosed here, for example, as previously described. A kit may, in some embodiments, include: a container adapted to contain a particulate aerogel material; the container comprising a plurality of openings sized to allow the particulate aerogel material to be transported therethrough; and instructions in any form that are provided in connection with the components of the kit in such a manner that one of ordinary skill in the art would recognize that the instructions are to be associated with the components of the embodiments described herein. In certain embodiments, the particulate aerogel contained within the container is not dispersed in a liquid. For instance, the instructions may include instructions for the use, modification, assembly, storage, or packaging of the components. In certain embodiments, the instructions include instructions for mixing, diluting, preserving, administering, and/or preparing compositions (e.g., particulate aerogel material, optional liquids, and the like) for use in association with the components of the kit. In some cases, the instructions may also include instructions for the use of the components or associated compositions, for example, for a particular use, e.g., for application to a handle. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

The exemplary systems and methods of this disclosure have been described in relation to methods of treating a skin irritation. However, to avoid unnecessarily obscuring the present disclosure, the preceding description omits a number of known structures and devices, and other application and embodiments. This omission is not to be construed as a limitation of the scopes of the claims. Specific details are set forth to provide an understanding of the present disclosure. It should however be appreciated that the present disclosure may be practiced in a variety of ways beyond the specific detail set forth herein.

Also, while the methods have been discussed and illustrated in relation to a particular sequence of events, it should be appreciated that changes, additions, and omissions to this sequence can occur without materially affecting the operation of the disclosed embodiments, configuration, and aspects.

A number of variations and modifications of the disclosure can be used. It would be possible to provide for some features of the disclosure without providing others.

Although the present disclosure describes components and functions implemented in the aspects, embodiments, and/or configurations with reference to particular standards and protocols, the aspects, embodiments, and/or configurations are not limited to such standards and protocols. Other similar standards and protocols not mentioned herein are in existence and are considered to be included in the present disclosure. Moreover, the standards and protocols mentioned herein, and other similar standards and protocols not mentioned herein are periodically superseded by faster or more effective equivalents having essentially the same functions. Such replacement standards and protocols having the same functions are considered equivalents included in the present disclosure.

The present disclosure, in various aspects, embodiments, and/or configurations, includes components, methods, processes, systems and/or apparatus substantially as depicted and described herein, including various aspects, embodiments, configurations embodiments, sub-combinations, and/or subsets thereof. Those of skill in the art will understand how to make and use the disclosed aspects, embodiments, and/or configurations after understanding the present disclosure. The present disclosure, in various aspects, embodiments, and/or configurations, includes providing devices and processes in the absence of items not depicted and/or described herein or in various aspects, embodiments, and/or configurations hereof, including in the absence of such items as may have been used in previous devices or processes, e.g., for improving performance, achieving ease and\or reducing cost of implementation.

The foregoing discussion has been presented for purposes of illustration and description. The foregoing is not intended to limit the disclosure to the form or forms disclosed herein. In the foregoing Detailed Description for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the description has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method for treating contact dermatitis, comprising the steps of:
dispensing a portion of particulate aerogel material from a container, wherein the portion of particulate aerogel is not dispersed in a liquid;
topically applying the portion of particulate aerogel material to an affected area of skin tissue;
allowing the portion of particulate aerogel material to remain on the affected area of skin tissue for a first period of time; and
removing the portion of particulate aerogel material from the area of skin tissue.

2. The method of claim 1, wherein the portion of particulate aerogel material has been rendered hydrophobic by reaction with a hydrophobe.

3. The method of claim 1, wherein the contact dermatitis is associated with sebum.

4. The method of claim 2, wherein removing the portion of particulate aerogel material from the affected area of skin tissue removes a portion of sebum from the affected area of skin tissue.

5. The method of claim 1, wherein the contact dermatitis is associated with urushiol.

6. The method of claim 5, wherein removing the portion of particulate aerogel material from the affected area of skin tissue removes a portion of urushiol from the affected area of skin tissue.

7. The method of claim 1, wherein the contact dermatitis is associated with a synthetic oil.

8. The method of claim 7, wherein removing the portion of particulate aerogel material from the affected area of skin tissue removes a portion of synthetic oil from the affected area of skin tissue.

9. The method of claim 1, wherein removing the portion of particulate aerogel material from the affected area of skin tissue removes a portion of skin irritant from the affected area of skin tissue.

10. The method of claim 1, wherein the first period of time is greater than or equal to one minute.

11. A method of treating a skin irritation, the method comprising:
dispensing a portion of particulate aerogel material from a container;
applying the portion of particulate aerogel material to a skin irritation area, the skin irritation area comprising a skin irritant and having a baseline skin irritation value, wherein:
the portion of particulate aerogel material engages at least the skin irritant of the skin irritation area to form a treatment mixture;
at least some of the skin irritant is removed from the skin irritation area by the portion of particulate aerogel material; and
the baseline skin irritation value is reduced by removal of the skin irritant.

12. The method of claim 11, wherein the skin irritant is associated with sebum.

13. The method of claim 11, wherein the skin irritant is associated with a dermatitis.

14. The method of claim 11, wherein the skin irritant is an urushiol.

15. The method of claim 11, wherein the skin irritant is a synthetic oil.

16. The method of claim 11, wherein the baseline skin irritation value is a measure of the proportion of skin irritant to a skin oil.

17. The method of claim 11, wherein the skin irritant is removed by removing a portion of the treatment mixture from the skin irritation area.

18. A method of treating an anticipated irritation to a skin area of an individual, the method comprising:
dispensing a portion of particulate aerogel material from a container; and
applying the portion of particulate aerogel material to the skin area, wherein:
the portion of particulate aerogel material is adapted to engage a skin irritant to form a treatment mixture; and
the portion of particulate aerogel material is adapted to be removed from the skin area.

19. The method of claim 18, wherein the skin irritant is an urushiol.

20. The method of claim 18, wherein the skin irritant is a synthetic oil.
